(19) United States
(12) Patent Application Publication
(10) Pub. No.: US 2002/0115211 A1
(43) Pub. Date: Aug. 22, 2002

(54) A METHOD FOR IN VITRO MATURATION OF HUMAN GAMETES

(75) Inventors: Svend LINDENBERG, Skodsborg (DK); Anne Lis MIKKELSEN, Frederiksberg (DK); Steven Dale SMITH, Bronshoj (DK); Kjell BERTHEUSSEN, Eidkjosen (NO)

Correspondence Address: Birch, Stewart, Kolasch & Birch, LLP, Birch, Stewart, Kolasch & Birch, LLP, 8110 Gatehouse Road, Suite 500 East, Falls Church, VA, 22040, US, mailroom@bskb.com, 703-205-8000, 703-205-8050

(73) Assignee: MEDI-CULT A/S, Jyllinge (DK)

(21) Appl. No.: 09/746,633

(22) Filed: Dec. 22, 2000

Related U.S. Application Data
(63) Continuation of application No. PCT/DK99/00345, filed on Jun. 22, 1999.

(30) Foreign Application Priority Data
Jun. 22, 1999 (DK) PCT/DK99/00345
Jun. 22, 1998 (DK) PA 1998 00885
Jun. 22, 1998 (US) 60/090,115

(51) Int. Cl. C12N 5/08

(52) U.S. Cl. 435/366

(57) ABSTRACT

### Abstract of Disclosure

The present invention relates to a method for in vitro maturation of a human gamete by culturing an immature human gamete in a chemically defined cell culture medium. The human gamete can be a spermatocyte or an oocyte. The maturation end point is metaII. The advantage of the described medium is a synchronised cumulus-, cytoand nuclear maturation completed within a period of 20 to 30 hours. The medium should preferably contain ATA (Aurin Tri Carboxylicacid) as an anti-apototic agent. The medium should preferably not contain BSA, HSA or other directly serum derived products or substances.

This application is a Continuation of PCT International Application No. PCT/DK99/00345 filed on June 22, 1999, which designated the United States and on which priority is claimed under 35 U.S.C. §120, the entire contents of which are hereby incorporated by reference.

### Background of the invention

The normal ovulating woman will recruit approx. 300 immature oocytes for each menstrual cycle. This recruitment takes place before the actual cycle. At the day of menstruation, around 20-30 immature oocytes will still be present. Normally, during a process of apoptosis all but one oocyte will die before ovulation. At day 5 - 10 approx. 10-15 immature oocytes will be present in their small follicles being 10-12 mm in diameter. Some still growing and some starting to undergo an apoptotic process.

Conventional in vitro fertilisation (IVF), treatment for special cases of severe male and female infertility, is based on retrieval of mature human oocytes followed by fertilisation of the mature oocytes with spermatozoa. The recruitment of human mature oocytes is accomplished through several complicated forms of hormone treatments, often with discomfort or risk for the woman involved. These hormone treatments will especially be a problem in the future, as IVF is increasingly offered to perfectly normal women in these programs due to their husbands´ poor sperm quality. Furthermore, this type of treatment will normally provide a pregnancy rate of 20% per started cycle. Because of the risk, discomfort and cost of the hormonal stimulation several other approaches have been tried during the years. In animalsin vitro maturation (IVM) has become an efficient method for producing oocytes for IVF, but until now recorded success rates for clinical human IVM have been low (Cha, Trounson, Barnes, Russel). One of the most simple ways to avoid hormonal stimulation has been not to stimulate with hormones at all. This treatment regimen, however, requires in vitro maturation of the gametes. The in vitro growth, development, and maturation is usually done in media comprising serum and serum derived products. As the gametes grow, develop and mature in vivo surrounded by serum, it has been natural to provide serum in the media. However, due to the potential transmission of disease from one mammal to another by use of serum or serum-derived products, the use of serum and serum-derived products has been greatly reduced over the recent years.

The research to find a chemically defined medium, wherein the non-fertilisable oocytes can mature in vitro to MF-II and then be capable of fertilisation and give rise to pregnancy after implantation into the female mammal, has been intensive over the last decade.

### DETAILED DESCRIPTION OF THE INVENTION

The present invention relates to a method for in vitro maturation of a human gamete by culturing an immature human gamete in a chemically defined cell culture medium. The human gamete could thus arise from a male as an immature spermatide or from a female as an immature prophase oocyte. With the technology of the present invention, it is possible to obtain immature human gametes from women or men in infertility treatment by aspirating and extracting these gametes from ovarian or testicular tissue. Furthermore, if a patient is diagnosed with cancer, testicular or ovarian tissue can be dissected out and frozen prior to initiation of treatment that might cause sterility such as cytostatic or radiation treatment, with the object of later extracting immature gametes from the frozen tissue. These immature gametes can then be finally matured in this chemically defined medium.

The method of the present invention will preferably start with immature or not fully matured gametes. In the woman the oocytes will be recognised as oocytes with a tight cumulus mass, no polar bodies or Germinal vesicles visible. These oocytes are readily recognised by a person involved in routine IVF-treatments as being immature oocytes. In the males all sperm precursors possessing a tail will be recognised as immature until all cytoplasm has been stripped of and a final normal spermatozoa is recognised. A normal mature spermatozoa is readily recognised by a person involved in IVF.

The advantages of starting with immature gametes as described above are several. A woman in treatment for infertility normally undergoes a complicated hormonal treatment for many days for gaining a sufficient number of mature prophase II oocytes for in vitro fertilisation. This hormonal treatment encompasses pain, discomfort, stress, and risk of ovarian hyperstimulation syndrome, a condition feared among patients and doctors. This hormonal therapy is instituted to rescue immature oocytes which will otherwise undergo apoptosis. Thus, these hormones are essential for allowing the immature oocytes to mature within the ovary in the substantial number needed for IVF. If no hormones are administrated only one oocyte will mature as seen in normal ovulating women. By releasing these immature oocytes from the ovary prior to initiation of the apoptotic processes and mature them further in a clinical defined medium, the woman can avoid the risk and discomfort associated with hormonal treatment and still have a sufficient number of mature Metaphase II (MF-II) oocytes for subsequent IVF treatment.

Oocyte maturation is the final stage of oocyte development that prepares for fertilisation and embryo development. It can be divided into two general processes: nuclear maturation and cytoplasmic maturation. Nuclear maturation is defined as the resumption of meiosis and progression to MF-II while cytoplasmic maturation is defined as the extragenomic changes that prepare the egg for activation, pronuclear formation, and early embryogenesis. Thus, by an immature female gamete is understood an ova that upon contact with a mature sperm cell will not complete the mitotic division and accept the genetic material from the sperm cell and form a fertilised cell. In one embodiment of the invention, the non-fertilisable, i.e. immature, female gamete, that is ova or oocyte, is a meiotic cell that is in a stage prior to germinal vesicle break-down (GVB), entrance into MF-I, and the folicle is antral or pre-antral.

By MF-II is understood an oocyte with 1 polar body, expanded cumulus complex and which has finally gone through a germinal vesicle break-down. These oocytes are readily recognised by a routine technician normally handling oocytes for IVF.

In humans it has been possible to produce oocytes whose nuclear maturation has progressed to MF-II, but which are incompetent to complete preimplantation development. The importance of cytoplasmic control over developmental competence has been described in the immature monkey oocyte. Using micromanipulation, ooplasm was removed from MF-II oocytes and injected into prophase I oocytes. Monkeys receiving the oocytes with cytoplasmic transfusion had a sevenfold increase in pregnancy rate compared to oocytes without ooplasm injection.

A special feature of importance in the present invention is that in the maturation in vitro by using the chemically defined medium, the developmental process of the human gamete up to MF-II is associated with a synchronised cumulus-, cytoplasm-, and nuclear maturation. The advantages of the synchronised maturation are clearly seen in the final pregnancy rate produced from these oocytes. By using this defined medium, not only the nuclear maturation is finalised normally, but also the cytoplasm and cumulus complexes are maturing in a normal sequence also seen in vivo. This is regarded as a crucial and important new finding that a chemically defined medium synchronise and optimize these processes. It is readily recognised that failure in synchronised maturation of these 3 components will jeopardize further development. Thus, in this invention the criteria for cell-stages is not only the maturation stage of the nucleus, but also the cytoplasma and cumulus expansion.

Another special feature of the present invention is that the maturation process is finished faster. Thus, as described in Example 2, culturing of the immature gamete from prophase to MF-II is completed within a period of 10 to 30 hours (such as 24 to 30 hours, i.e. 24 to 26 hours). This fast maturation minimises the risks of failure in cumulus expansion and cytoplasm disorders. Further, it minimises the exposure of the oocytes to longer culture time in vitro than necessary.

In a preferred embodiment of the present method, a chemically defined cell culture medium is used for the maturation process. The term "chemically defined medium" is to denote a medium without biologically extracted serum substances, and where all components and their concentration are known and described. The term "biologically extracted serum substances" includes substances such as immunoglobulins, but hormones such as growth hormones and gonadotrophins are not considered extracted from serum. If hormones or serum derived substances are to be added to the medium, recombinant hormones or serum derived substances are preferred. Preliminary results indicate that it has no effect on pregnancy rates to lower the content of Human Serum Albumin (HSA) in the medium from 5% to 0.5%. Thus, in one embodiment of the present invention, the contents of HSA, Bovine Serum Albumin (BSA) or other directly serum derived product is less than 0.5%, such as 0.4%, 0.3%, 0.2%, 0.1%, e.g. less than 0.05% and even less than 0.01%. In an alternative embodiment, the culture medium contains BSA or HSA obtained by recombinant methods, thereby eliminating the inter-mammal serum contact. In a much preferred embodiment the immature human gametes are cultured in a chemically defined medium without addition of directly serum-derived products or the patients' own serum or any other serum product derived directly from a mammal, such as a human or cattle.

The advantage of using a medium without biologically extracted serum substances is that the risk of transferring viruses or other pathogen or harmful particles to the medium and subsequently to the embryo is substantially reduced or non-existing. Furthermore, serum probably contains a factor, presently unknown, that inhibits the synchronised maturation of the nucleus, cytoplasma and cumulus expansion.

Thus, one aspect of the present invention relates to a method to avoid infection or contamination of a non-fertilisable gamete with known and/or unknown infectious agents (such as prions, viroids, virus, mycoplasma, bacteria, fungi) during in vitro maturation of the non-fertilisable gamete, by culturing the gamete in a medium without components originating from sources at least potentially containing infectious agents. In a preferred embodiment of that aspect, the method relates to avoiding contamination with toxic, teratogenic, carcinogenic, or mutagenic components.

The chemically defined medium should contain at least one factor that is capable of synchronising nuclear-, cytoplasma-, and cumulus cells maturation. In a preferred embodiment, the chemically defined medium contains synthetic lipid or lipid precursor, such as sterol or metabolically acceptable derivatives thereof. This could be cortisone. The advantages of using these compounds is to stabilise cell membranes, provide precursors for membrane building, and as a substance to be involved in local paracrine steroid production within the cumulus oocyte complex. Cortisone or derivatives can also be directly involved in stimulating and synchronising the final maturation of these immature oocytes.

The basic culture medium should be one that can both support the oocyte as well as its cumulus cells. It is well known in the art that addition of gonadotropins and/or steroid such as E₂ to the maturation medium enhances the fertilizability and/or developmental ability of e.g. cattle, monkey, and human oocytes. The addition of the gonadotrophins (FSH and hCG) to human IVM medium has been widely used but their optimal concentrations (or absolute necessity) have not been fully characterised. The cumulus cells can be considered a type of co-culture and as with other types of somatic cells, they generally require moderately high protein levels in the medium. It has been suggested that oocytes need to be primed with oestrogen in order to develop Ca++ oscillations. The medium of the present invention thus preferably contains oestrogens in concentrations of 0.1 to 10 µg/mL estradiol 17-β, e.g. 0.3 to 3 μg/mL estradiol 17-ß, preferably 1 μg/mL estradiol 17-β.

In a much preferred embodiment of the present invention, the chemically defined medium among other factors contains ATA (Aurin Tricarboxylic Acid) as an anti-apoptotic agent. The advantage of ATA is that it might provide optimal conditions to inhibit apoptotic processes otherwise deteriorating the oocyte maturation. Another advantage of the presence of ATA is that it allows the concentration of serum derived products, such as HSA or BSA to be lowered, such that the concentration of the serum derived products is zero.

In the present invention the term "apoptosis" should be understood as a controlled cell death, where the cell itself destroys its nuclear DNA, envisioned by DNA stand-breaks. The usage of an anti-apoptotic agent is preferred due to the fact that the oocyte retrieved is already engaged in an apoptotic process in the cumulus mass. When apoptosis starts in the oocyte-cumulus complex, this will signal the start of maturation. However, as this process progresses in the normal ovary, it will induce apoptosis in the oocyte. By removing the oocyte from the ovary after initiation of the apoptotic signal, which induces start of maturation, full development will take place in the chemically defined medium with e.g. ATA to stop further apoptosis.

In another embodiment of the present invention, the medium is used to culture any tissue or organ from a donor organism such that the tissue or organ acquire new functions. In a preferred embodiment the tissue or organ is transferred directly to the medium, that is the first medium to be used for the culture of the organ or donor is the medium of the present invention.

The chemically defined medium could be a medium as described in PCT/EP97/06721 hereby incorporated by reference. As an additive to the medium, a preferred additive is Medi-Cult SSR 4x, Medi-Cult SSR 4xa, Medi-Cult SSR 4xb, Medi-Cult SSR1or Medi-Cult SSR2 . As the basic medium, the preferred medium is Medi-Cult BBEM as described in Example 1. Insulin is a component of the above mentioned media. However, recent research has pointed out, that the presence of insulin in the culture medium has a negative effect on the chance for successful pregnancy. Therefore, in a preferred embodiment of the present invention, the culture medium is a culture medium as described above without insulin.

The chemically defined medium of the present invention with the contents as described is optimised to support the maturation of an non-fertilisable oocyte to MF-II and subsequent fertilisation and pregnancy after implantation into the female. In one embodiment of the invention, the pregnancy rate obtained when oocytes matured in the medium of the invention are used in a study of IVM-cycles is more than 10%, such as more than 13%, 15%, 18%, 20%, 21%, 22%, 23%, 24%, 25%, 26%, 27%, 28%, 29%, 30%, 31%, 32%, 33%, 34%, 35% or even more than 40%.

Apart from the contents of the medium, other factors are important in achieving this goal. These factors include the timing of the oocyte aspiration and the size of the follicles by the time of aspiration.

In a presently preferred embodiment of the present invention, the gamete is derived from ovarian follicles with a diameter of 8-12 mm. The advantage of such small follicles is that they are present in substantial numbers without severe hormonally treatment, they can be seen by ultrasound and an ultrasonically guided transvaginal puncture of the follicles is possible to perform in order to retrieve the oocyte.

An early apoptotic phase or an artificial plateau phase in the follicular growth may mimic the final preovulatory follicular maturation terms of developmental competence.

In vitro maturation of mammalian oocytes is not only related to growth of the follicle, but also to the size of the follicles and the oocytes. As seen in Example 2 the human oocyte appears to have a size dependant ability to resume meiosis and complete maturation. A decreased maturation rate and cleavage rate of oocytes obtained from follicles <8 mm is observed. These results suggest that capacity of human oocyte maturation is closely correlated with follicular maturation. As mentioned above, the maturing oocytes retrieved are in an early apoptotic phase. Thus, with increasing size of the oocytes the risk of obtaining oocytes in a late apoptotic phase, that is close to dead cells, increases. Based on these experiences, the preferred size of the oocytes retrieved is less than 12 mm.

The chemically defined medium can also be used for culturing immature sperm precursor cells from the testis. In severe male infertility, immature sperm cells are recovered either by needle biopsy or microsurgical techniques. The immature sperm cells and their surrounding Sertoli cell will because of the anti apoptotic activity in the medium benefit and mature the sperms. It is well known that apoptosis in the testis is the major factor to detoriate sperm function, specifically in the infertile man (E. Høst). Thus, the described technique is as follows: Extract sperm/Sertoli cell complexes from the infertile man. Let the extract be in culture for 24 hours and hereafter use the mature sperm for normal ICSI. The advantages of this is that the procedure of extracting spermatozoa from the man can take place days before the oocyte retrieval from the women. One can thus avoid oocyte aspiration if no sperms are present, and possibly better sperms will be retrieved.

In a preferred embodiment of the present invention the following steps are followed:- retrieval of ovarian follicles with a diameter of 8-12 mm by transvaginally ultrasound guided aspiration- culturing the retrieved ovarian oocytes (in prophase) using a chemically defined cell culture medium as described above to synchronise cumulus-, cytoplasm-, and nuclear maturations up to MF-II.

In some women it might be advantageous to initiate treatment on days 3, 4, and 5 in the menstrual cycle with hormones such as FSH followed by a discontinuation of treatment. In this treatment regimen, the blood level of estradiol in the woman might be monitored with the object of selecting maturating oocytes, indirectly measured by none growing (growth pause) follicles. Early apoptotic oocytes destined for becoming apoptotic are characterised in that they are easily detached from the ovary during the puncture of the follicles and they have a compact cumulus mass. Then by the time of a plateau or fall in estradiol level is observed, the retrieval of oocytes is performed.

However, in the preferred embodiment of the present invention the oocytes are aspirated from a woman, who has undergone prior hormonal treatment. One embodiment of the invention is a method for culturing an non-fertilisable oocyte to MF-II, such that the oocyte is capable of fertilisation, cleavage and successful implantation, in a chemically defined medium.

### References:

Lanzendorf SE, Gordon K, Mahony M, Boyd C, Neely B, Hodgen GD. The effect of coculture on the postfertilization development of in vitro-matured monkey oocytes. Fertil Steril 1996; 65:420-5.

Plachot M, Antoine JM, Alvarez S, Firmin C, Pfister A, Mandelbaum J, et al. Granulosa cells improve human embryo development in vitro. Human Reprod 1993;12:2133-40.

Freeman MR, Whitworth CM, Hill GA. Granulosa cell co-culture enhances human embryo development and pregnancy rate following in vitro fertilization. Hum Reprod 1995;2:408-14.

Durinzi KL, Saniga EM, Lanzendorf SE. The relationship between size and maturation in vitro in the unstimulated human oocyte. Fertil Steril 1995;63:404-6.

Flood J, Chillik CF, van Uem JFHM, Iritani A, Hodgen GD. Ooplasmatic transfusion: prophase germianl vesicle oocytes made developmentally competent by microinjection of methase II egg cytoplasma. Fertil Steril 1990;53:1049-54.

Sirard MA, Blondin P. The cow as a model for oocyte maturation in vitro.Abstract no PS-10-3. Journal of Assisted Reproduction and Genetics 1997; 14 (suppl) 19S.

Schramm RD, Bavister BD. Effects of granulosa cells and gonadotrophins on meiotic and developmental competance of oocytes in vitro in non-stimulated rhesus monkeys. Human Reprod 1995;10:887-95.

Schramm RD , Bavister BD.Granulosacells from follicle stimulating hormone primed monkeys enhance the developmental competance of in-vitro matured oocytes from non-stimulated rhesus monkeys. Human Reprod 1996;11:1698-1702.

Schramm RD , Bavister BD. FSH-priming of Rhesus monkeys enhances meiotic and developmental competence of oocytes in vitro . Biol Reprod 1994; 51:904 12.

Cha KY, Koo JJ, Ko JJ, Choi DH, Han SY, Yoon TK. Pregnancy after in vitro fertilization of human follicular oocytes collected from nonstimulated cycles, their culture in vitro and their transfer in a donor oocyte program. Fertil Steril 1991;55:109-13.

Russell JB, Knezevich KM, Fabian K, Dickson JA: Unstimulated immature oocyte retrieval: early versus midfollicular endometrial priming. Fertil Steril 1997;67:616-20.

Barnes FL, Kausche AK, Tiglias J, Wood C, Wilton L, Trounson A. Production of embryos from in vitro-matured primary oocytes. Fertil Steril 1996;65:1151-6.

Trounson A, Wood C, Kaunsche A. In vitro maturation and the fertilization and developmental competance of oocytes recovered from untreated polycystic ovarian patients. Fertil Steril 1994;62:353-62.

Cha KY, Chung HM, Han SY, Yoon TK, Oum KB, Chung MK. Successful in vitro maturation, fertilization and pregnancy by using immature follicular oocytes collected from unstimulated polycystic ovarian syndrome patients. Fertil Steril Abstract O-044;1996:Supl.S23.

Morgan PM, Warikoo PK, Bavister BV. In vitro maturation of ovarian oocytes from unstimulated rhesus monkeys: assessment of cytoplasmatic maturity by embryonic development after in vitro fertilization. Biology of Reproduction 1991;45:89-93.

Wynn P, Krapez J, Picton HM, Rutherford AJ and Gosden RG. Randomized study of oocytes matured in vitro after collection from unstimulated or mildly stimulated patients. Abstract .........1997;..:13-14.

Gomez E, Tarin JJ, Pellllicer A. Oocyte maturation in humans: the role of gonadotropins and growth factors. Fertil Steril 1993; 60:40-6.

Eppig JJ, Schultz RM, O`Brien M, Chewnel F. Relationship between the developmental programs controlling nuclear and cytoplasmic maturation of mouse oocytes. Dev Biol 1994; 164:1-9.

Tsuji K, Sowa M, Nakano R. Relationship between Human oocyte maturation and different follicular sizes. Biol Reprod 1985; 32:413-7.<Fauser BCJM. Follicular development and oocyte maturation in hypogonadotropic women employing recombinant follicle-stimulation hormone: the role of oestradiol. Human Reproduction Update 1997;3:101-8.

Tesarik J, Mendoza. Direct non-genomic effects of follicular steroids on maturing human oocytes: oestrogen versus androgen antagonism. Human Reproduction Update 1997;3:95-100.

Anderiesz C, Trounson AO. The effect of testosterone on the maturation and developmental capacity of murine oocytes in vitro. Hun Reprod 1995;10:2377-81.

Saiki K, Hoshi M, Liebfriied-Rutledge ML, First. In vitro fertilization and development of bovine oocytes matured in serum-free medium. Biol Reprod 1991;44:256-60.

Zhang X, Xerafi A, Wong J, Armstrong D, Khami F. Human menopausal gonadotropin during in vitro maturation of human oocytes retrieved form small follicles enhanced in vitro fertilization and cleavage rate. Fertil Steril 1993;58:850-3.

E. Høst S. Lindenberg 1998 Acta Scand. Gynecol. Obstet. Accepted.

### EXAMPLES

It is to be understood that the examples described below are illustrative of embodiments of the present invention, and the invention is not intended to be so limited.

### Example 1 Culturing of the immature gamete to metaphase II

Immature oocytes are aspirated transvaginally with a 17 g Cook needle (Cook, Australia) under low aspiration pressure. Follicular aspirates are collected into tubes or syringes containing warmed Flushing Medium (Medi-Cult, Denmark).

All manipulations are carried out at 37°C. Follicular aspirates are filtered (Falcon 1060) to remove erythrocytes and small cellular debris. The retained cells are resuspended in equilibrated BBEM (Medi-Cult, Denmark) with bicarbonate and HEPEs buffers and then oocytes are isolated under a stereomicroscope and washed twice in BBEM (pH 7.2-7.4, mosmol/kg: 285±8, Modified EBS with lactate, MEM non essential amino acids, 2.5 mM HEPES, 0.1 mM Taurine, 200 mM Ultra-glutamine, 0.2 mM sodium pyruvate, 0.5 mM d-glucose, 0.8 mM MgSo4 anhydrous, 3.6 mM Ca-lactate, 1mM NaH2PO4, 5.4 mM K2SO4, 110 mM NaCl, 1 ml/l SSR4xb (Medi-Cult, Denmark). Immature oocytes are incubated in BBEM in 5% CO₂ and air at 37°C for 2 h before being transferred into IVM medium.

The IVM medium consists of BBEM supplemented with SSR4x 1:1000 (Medi-Cult, Denmark), 0.075 IU/mL recombinant human FSH, 0.5 IU/mL hCG (both from Serono, Denmark), 1 µg/mL estradiol 17-β (Sigma, Denmark), and 5% HSA (except for the oocytes from patient 1 which are cultured in 0.5% HSA). Oocytes are cultured singly in 25 µL drops of IVM medium under paraffin oil at 37°C in 5% CO2 and humidified air. During the growth cumulus expansion is observed as a sign of healthy maturing oocytes. Images are recorded at appoximately 24 h and again at either 36 or 48 h of culture, and the number of cells in MF-II are counted.

Oocytes are denuded with hyaluronidase (IVF Science, Sweden) and mechanical pipetting. Motile sperm are prepared by Puresperm^{TM} (Cryos, Denmark) gradient separation or by swim-up. For ICSI denuded oocytes are placed individually into 5 µL drops of sperm prep medium (Medi-Cult, Denmark) and 2 µL of sperm suspension is placed into a 10 µL drop of PVP (IVF Science, Sweden). All metaphase II oocytes are inseminated by ICSI and then placed into 10 µL drops of BBEM and cultured in 5% CO₂ and humidified air at 37°C. Approximately 10-20 h after insemination oocytes are examined at 300X for the presence of pronuclei as a measure of successful fertilization. Embryos are cultured to day 2 or 3 (day 0 = day of insemination) at which time suitable embryos (maximum of 2) are replaced into the women. Suitable embryos are those that are cleaved. The suitable embryos are scored on a scale from 1 (best) to 4 (worst) prior to replacement.

Oocytes from patient (pt) 1-3 are cultured in TCM-199 without SSR4x instead of BBEM with SSR4x for 36h. Oocytes from pt A-C are cultured for 26h.

| Pt. | No of Oocytes | Cumulus expansion | MF-II | Fertilized | Cleaved | Score |
|---|---|---|---|---|---|---|
| 1 | 5 | 0 | 2 | 2 | 2 | 2.1 |
| 2 | 3 | 3 | 2 | 1 | 1 | 2.2 |
| 3 | 3 | 3 | 3 | 2 | 2 | 2.1 |
| A | 1 | 1 | 1 | 1 | 1 | 3.0 |
| B | 2 | 2 | 2 | 2 | 2 | 2.1 |
| C | 8 | 6 | 6 | 4 | 3 | 2.1 |

These results as presented in Table 1 show that using the BBEM medium, metaphase II is obtained as well as in the other medium, but in a shorter time.

### EXAMPLE 2 The impact of FSH priming

Eighteen patients are recruited among PCO patients referred for IVF. Included in the study are all women with typical PCO patterns at ultrasound, which means more than ten follicles in one plane located subcapsulary in each ovary. Excluded are all patients with body weight more than 20% of normal for the same height and women with contradictions for pregnancy. Also excluded are patients with elevated prolactin and FSH value (>10 IU) on cycle day 3. The study is approved by the local ethical committee. All women particiin the study after written consent.

The women are randomly allocated to three groups according to their entrance in the study. Group I (n=6 cycles) receive no stimulation, Group II (n=6 cycles) receive rec-FSH from day 3 and given from 3 to 7 days, aspiration is performed within 24 hours after the last injection. In group III (n=8 cycles), stimulation with rec-FSH (Gonal-F) as in group II, but oocyte recovery is performed 48-72 hours after the last injection.

Immature oocyte collection is performed between cycle day 7 and 13 by use of an aspiraneedle from Cook Ltd described by Carl Wood. The aspiration pressure is reduced to 100 mmHg on the assumption that the immature cumulus or egg may be more susceptito mechanical injury, or the needle in a small follicle may directly damage the egg if the egg is sucked forcefully from a small volume of fluid into the end of the needle. The follicles are punctured. After aspiration the needle is flushed with Earles Balanced Salt solution with Hepes and bicabonate buffers plus heparin (100IU/ml) as described by Trounson et al.

The follicular aspirates are transferred in tubes to the laboratory and washed on an emfilter with a pore size of 70 μm. Erythrocytes and other small cells are washed through the filter and oocytes and larger fragments of cells are collected in Petri culture dishes. The immature oocytes are identified and graded based on the presence or abof cumulus cells as ether complete multilayered, sparse or nude.

Oocytes are matured in tissue culture medium 199 (TCM 199; Sigma) supplemented with Sodiumpyruvate 0.3mM, rec-FSH 0.075IU/ml (Gonal-F; Serono), hCG 0.05 IU/ml (Profasi; Serono), and albumin 1%. Later the medium is supplemented with oestradiol 1μg/ml, an increased concentration of HSA (5%) or serum from the patient (10%) instead of albumin.

Fertilization with ICSI is performed on all Metaphase II oocytes and the oocytes are checked for the presence of two pronuclei the day after insemination and cleavage of the 2PN-oocytes is registered on day 2 after insemination.

The statistical methods include Student´s t-test. A two-tailed p<0.05 is considered statistisignificant.

The clinical characteristics of the three groups is shown in Table 2. There are no differin the mean age of the women and indications for IVF between the three groups. Anovulatory and ovulatory PCO patients are evenly distributed between the groups.

ResultsGroup 1.

In 6 patients without hormonal stimulation the oocyte pick-ups are performed day 7 - 9 when the follicles are 2 to 10 mm in diameter. We did not recover any oocytes from 2 pafrom the remaining 4 we obtained 28 oocytes, of which 13 were used for in vitro maturation. The rest were apoptotic or without cumulus cells.

Seven oocytes matured and were used for ICSI, 5 cleaved and 3 were transferred in two patients. These oocytes were obtained from follicles at 8-10 mm.

Group 2.

Six patients had stimulation with rec-FSH and aspiration the day after the last injection. In these patients 28 oocytes were recovered and 26 submitted to IVM. 6 matured to MF-II, one oocyte cleaved and was transferred. Also in this group we observed that oocytes from small follicles (<8 mm) did not mature to MF-II.

Group 3.

Eight patients received stimulation with rec-FSH. Oocyte aspiration was performed 48-72 hours after the last injection when the follicles were 10 - 14 mm in diameter. We obtained 40 oocytes and 35 were used for IVM, 29 matured to MF-II and 20 cleaved. In two pawith 48 hours delay between the last injection and aspiration late maturation was observed. MF-II oocytes were seen after 56 hours, they were injected and fertilisation and cleavage was obtained. Transfer was possible in all patients in this group. One pregnancy was obtained. The patients had 9 immature oocytes retrieved after stimulation for 3 days with 150 IU Gonal-F. Six oocytes matured and 5 cleaved. Two four cell embryos were transferred and 2 were cryopreserved.

Significantly more oocytes were enclosed by cumulus and could be used for in vitro matuafter FSH stimulation. Maturation rate and cleavage rate were significantly reduced in group 2 with FSH priming, compared to unstimulated oocytes (group 1) and FSH primed oocytes aspirated with a delay after FSH injection(group 3). The maturation rate was significantly increased in group 3 compared to group 1, but no difference in cleavage rate was observed (Table 3).

Independently of FSH stimulation we observed a decreased maturation rate and cleavage rate of oocytes obtained from follicles <8 mm. In total 12 oocytes were obtained from these follicles and only one matured to MF-II.

Table 2 The clinical characteristics of the three groups

| | Group 1no stimulation | Group 2rec-FSH stimulation and aspiration without delay | Group 3rec-FSH stimulation and aspiration after 72 hours delay |
|---|---|---|---|
| No cycles | 6 | 6 | 8 |
| PCOPreviously IUI | 4 | 3 | 4 |
| Previously IVF | 2 | 3 | 4 |
| Age of the patientsMedian and range (years) | 3027-35 | 3127-37 | 3125-35 |
| Aspiration dayMedium and range | 87-9 | 77-11 | 109-13 |
| Size of the follicles (mm) | 2-10 | 5-13 | 10-14 |
| No. Ampoules rec.FSHMedium and range | | 66-20 | 106-12 |

Table 3 The no. of oocytes obtained for IVM, maturation rate and cleavage rate in the three groups

| | No patients | no oocytesaspirated | no oocytes for IVM | MF-IIno (%) | cleavageno (%) | Transferno | Pregnancyno |
|---|---|---|---|---|---|---|---|
| group 1 | 6 | 28 | 13¹ | 7 (54)² | 5 (71)³ | 2 | 0 |
| group 2 | 6 | 28 | 26¹ | 6 (23) ² | 1 (16) ³ | 1 | 0 |
| group 3 | 8 | 40 | 35¹ | 29 (82) ² | 20 (69) ³ | 8 | 1 |

1: Groups 1 and 2, and groups 2 and 3 are significantly different.

2: Percentage of cumulus enclosed oocytes. Significantly difference between the three groups is found.

3: Percentage of mature MF-II oocytes. Group 2 differs significantly from group 1 and group 3.

Example 3: contents of Medi-Cult media

SSR1 contains 1000x pr litre Table 4 SSR1 contents

| | |
|---|---|
| Compound | 1000x per litre |
| Trisodium citrate-dihydrate | 7.35 g |
| Citric acid | 3.15 g |
| Pluronic F-68 | 20 g |
| Aurintricarboxylic acid (ATA) | 1.27 g |
| Ethylenediaminetetraacetic acid Fe(III)-Na-chelate dihydrate | 1.20g |
| EDTA-Na2 (triplex III) | 0.372g |
| Trace elements (100 000x) | |
| EDTA-Na2; | 5.211 g |
| Trisodium citrate-dihydrate | 0.294g |
| Zinc sulfate-heptahydrate | 2.875 g |
| Copper(II)sulfate-pentahydrate | 0.499 g |
| Manganese sulfate, H₂0 | 0.017 ml |
| Nikkel(II)-nitrat-hexahydrat | 0.0058 g |
| Ammoniumaluminiumsulfate-12-hydrate | 0.092 g |
| Potassium-chromium sulfate-pentahydrate | 0.05 g |
| Cobalt(II)-chloride-hexahydrate | 0.048 g |
| Selenium dioxide | 0.111 g |

Table 5 SSR2 contents

| | |
|---|---|
| Compound (1000x) | pr litre |
| Trisodium citrate-dihydrate | 7.35 g |
| Citric acid | 3.15 g |
| Pluronic F-68 | 20 g |
| Aurintricarboxylic acid (ATA) | 1.27 g |
| Ethylenediaminetetraacetic acid Fe(III)-Na-chelate dihydrate | 1.20g |
| EDTA-Na2 (triplex III) | 0.372g |
| HCl , 1N, | 5 g |
| Human insulin recombinant (NOVO) | 0.50 g |
| Trace elements (100 000x) | |
| EDTA-Na2; | 5.211 g |
| Trisodium citrate-dihydrate | 0.294g |
| Zinc sulfate-heptahydrate | 2.875 g |
| Copper (II)sulfate-pentahydrate | 0.499 g |
| Manganese sulfate, H₂0 | 0.017 ml |
| Nickel(II)-nitrate-hexahydrate | 0.0058 g |
| Ammoniumaluminiumsulfate-12-hydrate | 0.092 g |
| Potassium-chromium sulfate-pentahydrate | 0.05 g |
| Cobalt(II)-chloride-hexahydrate | 0.048 g |
| Selenium dioxide | 0.111 g |

Table 6 SSR4x contents

| | |
|---|---|
| Compound (1000x) | pr litre |
| Trisodium citrate-dihydrate | 7.35 g |
| Citric acid | 3.15 g |
| Pluronic F-68 | 20 g |
| Aurintricarboxylic acid (ATA) | 1.27 g |
| Ethylenediaminetetraacetic acid Fe(III)-Na-chelate dihydrate | 1.20g |
| EDTA-Na2 (triplex III) | 0.372g |
| HCl , 1N, | 5 g |
| Human insulin recombinant (NOVO) | 0.50 g |
| Ethanol | 1000 ml |
| Cholesterol | 2.0 g |
| Polyvinyl pyrrolidone (PVP 10) | 250 g |
| Acetic acid (glacial) 100% | 6.0 ml |
| Ethanolamine | 1.2 ml |
| Trace elements (100 000x) | |
| EDTA-Na2; | 5.211 g |
| Trisodium citrate-dihydrate | 0.294g |
| Zinc sulfate-heptahydrate | 2.875 g |
| Copper(II)sulfate-pentahydrate | 0.499 g |
| Manganese sulfate, H₂0 | 0.017 ml |
| Nickel(II)-nitrate-hexahydrate | 0.0058 g |
| Ammoniumaluminiumsulfate-12-hydrate | 0.092 g |
| Potassium-chromium sulfate-pentahydrate | 0.05 g |
| Cobalt(II)-chloride-hexahydrate | 0.048 g |
| Selenium dioxide | 0.111 g |

Table 7 SSR4xa contents

| | |
|---|---|
| Compound (1000x) | pr litre |
| Trisodium citrate-dihydrate | 7.35 g |
| Citric acid | 3.15 g |
| Pluronic F-68 | 20 g |
| Aurintricarboxylic acid (ATA) | 1.27 g |
| Ethylenediaminetetraacetic acid Fe(III)-Na-chelate dihydrate | 1.20g |
| EDTA-Na2 (triplex III) | 0.372g |
| HCl , 1N, | 5 g |
| Human insulin recombinant (NOVO) | 0.50 g |
| Ethanol | 1000 ml |
| Polyvinyl pyrrolidone (PVP 10) | 250 g |
| Acetic acid (glacial) 100% | 6.0 ml |
| Ethanolamine | 1.2 ml |
| Trace elements (100 000x) | |
| EDTA-Na2; | 5.211 g |
| Trisodium citrate-dihydrate | 0.294g |
| Zinc sulfate-heptahydrate | 2.875 g |
| Copper(II)sulfate-pentahydrate | 0.499 g |
| Manganese sulfate, H₂0 | 0.017 ml |
| Nickel(II)-nitrate-hexahydrate | 0.0058 g |
| Ammoniumaluminiumsulfate-12-hydrate | 0.092 g |
| Potassium-chromium sulfate-pentahydrate | 0.05 g |
| Cobalt(II)-chloride-hexahydrate | 0.048 g |
| Selenium dioxide | 0.111 g |

Table 8 SSR4xb contents

| | |
|---|---|
| Compound (1000x) | pr litre |
| Trisodium citrate-dihydrate | 7.35 g |
| Citric acid | 3.15 g |
| Pluronic F-68 | 20 g |
| Aurintricarboxylic acid (ATA) | 1.27 g |
| Ethylenediaminetetraacetic acid Fe(III)-Na-chelate dihydrate | 1.20g |
| EDTA-Na2 (triplex III) | 0.372g |
| HCl , 1N, | 5 g |
| Human insulin recombinant (NOVO) | 0.50 g |
| Ethanol | 1000 ml |
| Cholesteryl acetate | 2.0 g |
| Polyvinyl pyrrolidone (PVP 10) | 250 g |
| Acetic acid (glacial) 100% | 6.0 ml |
| Ethanolamine | 1.2 ml |
| Trace elements (100 000x) | |
| EDTA-Na2; | 5.211 g |
| Trisodium citrate-dihydrate | 0.294g |
| Zinc sulfate-heptahydrate | 2.875 g |
| Copper(II)sulfate-pentahydrate | 0.499 g |
| Manganese sulfate, H₂0 | 0.017 ml |
| Nickel(II)-nitrate-hexahydrate | 0.0058 g |
| Ammoniumaluminiumsulfate-12-hydrate | 0.092 g |
| Potassium-chromium sulfate-pentahydrate | 0.05 g |
| Cobalt(II)-chloride-hexahydrate | 0.048 g |
| Selenium dioxide | 0.111 g |

Table 9 SSR3 contents

| | |
|---|---|
| Compound (1000x) | pr litre |
| Trisodium citrate-dihydrate | 7.35 g |
| Citric acid | 3.15 g |
| Pluronic F-68 | 20 g |
| Aurintricarboxylic acid (ATA) | 1.27 g |
| Ethylenediaminetetraacetic acid Fe(III)-Na-chelate dihydrate | 1.20g |
| EDTA-Na2 (triplex III) | 0.372g |
| HCl , 1N, | 5 g |
| Human insulin recombinant (NOVO) | 0.50 g |
| Ethanolamine | 1.2 ml |
| Trace elements (100 000x) | |
| EDTA-Na2; | 5.211 g |
| Trisodium citrate-dihydrate | 0.294g |
| Zinc sulfate-heptahydrate | 2.875 g |
| Copper(II)sulfate-pentahydrate | 0.499 g |
| Manganese sulfate, H₂0 | 0.017 ml |
| Nickel(II)-nitrate-hexahydrate | 0.0058 g |
| Ammoniumaluminiumsulfate-12-hydrate | 0.092 g |
| Potassium-chromium sulfate-pentahydrate | 0.05 g |
| Cobalt(II)-chloride-hexahydrate | 0.048 g |
| Selenium dioxide | 0.111 g |

Claims

1. A method for in vitro maturation and fertilization of a human oocyte characterized by culturing an immature human oocyte in a chemically defined cell culture medium, followed by fertilization.

2. A method according to claim 1 wherein the immature oocyte is in the prophase.

3. A method according to claim 1, wherein the oocyte after fertilization is transferred to the uterus.

4. A method according to claim 1, wherein the oocyte is cultured for a period of 10-30 hours before fertilization.

5. A method according to claim 4, wherein the oocyte is cultured for a period of 20-30 hours before fertilization.

6. A method according to claim 1, wherein the chemically defined cell culture mecontains at least one factor that is capable of synchronising nuclear-,cytoplasma-, and cumulus cells maturation.

7. A method according to claim 1, wherein the chemically defined medium contains lipid or lipid precursor, such as sterol or functionally equivalents derivatives thereof.

8. A method according to claim 1 wherein the chemically defined medium among other factors contains ATA (Aurin Tricarboxylic Acid).

9. A method according to claim 1 wherein the chemically defined medium is a meas described in PCT/EP97/06721.

10. A method according to claim 1 wherein the chemically defined medium contains Medi-Cult SSR 4x, Medi-Cult SSR 4xa, Medi-Cult SSR 4xb, Medi-Cult SSR2, or Medi-Cult SSR3.

11. A method according to claim 1 wherein the chemically defined medium is based on Medi-Cult BBEM.
